(19) United States
(12) Patent Application Publication
(10) Pub. No.: US 2008/0154094 A1
(43) Pub. Date: Jun. 26, 2008

(54) INSERTION PART OF ENDOSCOPE, ENDOSCOPE WITH INSERTION PART, AND FORMING METHOD OF FIXING STRUCTURE FOR BENDING PORTION OF INSERTION PART OF ENDOSCOPE

(76) Inventor: Ichiro NAKAMURA, Kokubunji-shi (JP)

Correspondence Address: OSTROLENK FABER GERB & SOFFEN, 1180 AVENUE OF THE AMERICAS, NEW YORK, NY, 100368403, US

(21) Appl. No.: 11/847,436

(22) Filed: Aug. 30, 2007

(30) Foreign Application Priority Data
Sep. 1, 2006 (JP) 2006-238039

(51) Int. Cl. A61B 1/005; B29C 59/00

(52) U.S. Cl. 600/129; 264/446

(57) ABSTRACT

A vending portion between the distal end of a flexible tube portion and a tip portion in an insertion part of an endoscope includes a tubular vending structure, a rubber tube having both end portions covering the distal end neighboring region of the outer surface of the tube portion and the base end neighboring region of the outer surface of the tip portion, and fixing structures fixing the both end portions of the rubber tube to the distal end neighboring region and the base end neighboring region in a water-tight manner. The fixing structure for at least one of the both end portions of the rubber tube includes a string member including thermoplastic and winding many times in an adjoining manner on the outer surface of the at least one portion, the outer side areas of the windings of the string member being melted and fixed to each other.

### CROSS-REFERENCE TO RELATED APPLICATIONS

This application is based upon and claims the benefit of priority from prior Japanese Patent Application No. 2006-238039, filed Sep. 1, 2006, the entire contents of which are incorporated herein by reference.

### BACKGROUND OF THE INVENTION

1. Field of the Invention

The present invention relates to an insertion part of an endoscope, an endoscope with the insertion part, and a forming method of a fixing structure for a bending portion of the insertion part of the endoscope.

2. Description of the Related Art

For example, a flexible endoscope for medical applications has an operating part and a slender insertion part configured to extend from the operating part and to be inserted into a body cavity of a living organism. The insertion part includes a slender flexible tube portion having a proximal end directly connected to the operating part, a tubular bending portion having one end connected to the distal end of the flexible tube portion, and a tip portion having a base end connected to the other end of the bending portion.

The bending portion includes a tubular bending structure that can be bent in a radius smaller than that of the flexible tube portion and an exterior rubber tube which covers an outer peripheral surface of the bending structure. A conventional bending structure includes, for example, a bending tube in which concentrically arranged multiple rings are linked bendably in four directions (cruciform directions) as a whole, a knitted pipe which is mounted on an outer peripheral surface of the bending tube, and an outer peripheral surface of the knitted pipe is covered by the exterior rubber tube. Wires are inserted through each of the multiple rings at 4 positions separated from each other at even intervals in a circumferential direction of each ring, and one end of each wire is connected to the tip portion. The other ends of the four wires are connected to two bending portion operating knobs of the operating part through the slender flexible tube portion, and by operating the two bending portion operating knobs, the bending portion with the tip portion can be bent in four directions (cruciform directions) with respect to the distal end of the flexible tube portion.

One end portion of the exterior rubber tube is put on a distal end neighboring region being adjacent to the distal end on an outer peripheral surface of the flexible tube portion, and is watertightly fixed to the neighboring region of the distal end by a fixing structure. The other end portion of the exterior rubber tube is put on a base end neighboring region being adjacent to the base end on the outer peripheral surface of the tip portion, and is watertightly fixed to the base end neighboring region by a fixing structure.

In Jpn. Pat. Appln. KOKAI Publication No. 6-319677, one example of the fixing structure is disclosed. This fixing structure includes strings made of, for example, silk, nylon, polyimide, and the like, and wound in layers around the both end portions of the exterior rubber tube that cover the base end neighboring region of the outer peripheral surface of the tip portion and the distal end neighboring region of the outer peripheral surface of the flexible tube portion, and adhesives applied to outer peripheries of the layers of the wound strings.

A predetermined amount of the adhesive must be applied to a predetermined area, and the adhesive applying work is conducted by skilled workers with relatively long time.

In Jpn. Pat. Appln. KOKAI Publication No. 2000-41937, another example of the fixing structure is disclosed. This fixing structure includes strings made of, for example, silk, nylon, polyimide, and the like, and wound in layers around the both end portions of the exterior rubber tube that put on the base end neighboring region of the outer peripheral surface of the tip portion and the distal end neighboring region of the outer peripheral surface of the flexible tube portion, and an outer shell made of thermoplastic resin covered on, and fused to the outer peripheries of the layers of the wound strings.

The fusing of the outer shell of thermoplastic resin is performed as follows. The outer periphery of the layer of the wound string is covered with the outer shell of thermoplastic resin, and an outer peripheral surface of the outer shell is covered with a heat-shrinkable tube. Next, hot air is blew on the heat-shrinkable tube to make the heat-shrinkable tube being heat-shrunk and at the same time to melt the outer shell of thermoplastic resin so as to be fused on the outer periphery of the layer of the wound string. At last, the heat-shrunk tube is removed from the outer periphery of the fused outer shell of thermoplastic resin after heat is radiated.

The above described fusing work of the outer shell of thermoplastic resin requires many working steps and thus increases a manufacturing cost of the insertion part.

### BRIEF SUMMARY OF THE INVENTION

An insertion part of an endoscope according to one aspect of the present invention is configured to extend from an operating part in the endoscope and to be inserted into a body cavity of a living organism. The insertion part comprises: a slender flexible tube portion having a proximal end and a distal end, the proximal end being connected to the operating part; a bending portion having one end and the other end, the one end being connected to the distal end of the flexible tube portion; and a tip portion having a base end and a tip end, the base end being connected to the other end of the bending portion. The bending portion includes: a tubular bending structure bendable to a radius smaller than the flexible tube portion; an exterior rubber tube which has one end portion put on a distal end neighboring region being adjacent to the distal end on an outer peripheral surface of the flexible tube portion and the other end portion put on a base end neighboring region being adjacent to the base end on an outer peripheral surface of the tip portion, and which covers an outer peripheral surface of the bending structure; and fixing structures which watertightly fix the one end portion and the other end portion of the exterior rubber tube to the distal end neighboring region of the outer peripheral surface of the flexible tube portion and the base end neighboring region of the outer peripheral surface of the tip portion. The fixing structure for at least one of the one end portion and the other end portion of the exterior rubber tube includes a string member containing thermoplastic resin and wound in multiple turns to be adjacent to each other on the outer peripheral surface of the at least one of the one end portion and the other end portion of the exterior rubber tube, and at least outer regions directed to an outside in the multiple turns of the string member on the outer peripheral surface of the at least one of the one end portion and the other end portion of the exterior rubber tube are melted and firmly fused to each other.

An endoscope according to one aspect of the present invention comprises: an operating part; and an insertion part configured to extend from the operating part and to be inserted into a body cavity of a living organism. The insertion part comprises: a slender flexible tube portion having a proximal end and a distal end, the proximal end being connected to the operating part; a bending portion having one end and the other end, the one end being connected to the distal end of the flexible tube portion; and a tip portion having a base end and a tip end, the base end being connected to the other end of the bending portion. The bending portion includes: a tubular bending structure bendable to a radius smaller than the flexible tube portion; an exterior rubber tube which has one end portion put on a distal end neighboring region being adjacent to the distal end on an outer peripheral surface of the flexible tube portion and the other end portion put on a base end neighboring region being adjacent to the base end on an outer peripheral surface of the tip portion, and which covers an outer peripheral surface of the bending structure; and fixing structures which watertightly fix the one end portion and the other end portion of the exterior rubber tube to the distal end neighboring region of the outer peripheral surface of the flexible tube portion and the base end neighboring region of the outer peripheral surface of the tip portion. The fixing structure for at least one of the one end portion and the other end portion of the exterior rubber tube includes a string member containing thermoplastic resin and wound in multiple turns to be adjacent to each other on the outer peripheral surface of the at least one of the one end portion and the other end portion of the exterior rubber tube, and at least outer regions directed to an outside in the multiple turns of the string member on the outer peripheral surface of the at least one of the one end portion and the other end portion of the exterior rubber tube are melted and firmly fused to each other.

In a forming method of a fixing structure for a bending portion of an insertion part of an endoscope, according to one aspect of the present invention: the endoscope comprises an operating part, and an insertion part configured to extend from the operating part and to be inserted into a body cavity of a living organism; the insertion part comprises a slender flexible tube portion having a proximal end and a distal end, the proximal end being connected to the operating part, a bending portion having one end and the other end, the one end being connected to the distal end of the flexible tube portion, and a tip portion having a base end and a tip end, the base end being connected to the other end of the bending portion; and the bending portion includes a tubular bending structure bendable to a radius smaller than the flexible tube portion, an exterior rubber tube which has one end portion put on a distal end neighboring region being adjacent to the distal end on an outer peripheral surface of the flexible tube portion and the other end portion put on a base end neighboring region being adjacent to the base end on an outer peripheral surface of the tip portion, and which covers an outer peripheral surface of the bending structure, and fixing structures which watertightly fix the one end portion and the other end portion of the exterior rubber tube to the distal end neighboring region of the outer peripheral surface of the flexible tube portion and the base end neighboring region of the outer peripheral surface of the tip portion. The forming method comprises winding a string member containing thermoplastic resin in multiple turns to be adjacent to each other on the outer peripheral surface of the at least one of the one end portion and the other end portion of the exterior rubber tube, and melting at least outer regions directed to an outside in the multiple turns of the string member on the outer peripheral surface of the at least one of the one end portion and the other end portion of the exterior rubber tube, and firmly fusing to each other.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic perspective view of the whole of a first embodiment of an endoscope according to the present invention;

FIG. 2 is a schematic perspective view showing a distal end of a slender flexible tube portion of an insertion part of the endoscope of FIG. 1, a bending portion whose one end is connected to the distal end, and a tip portion connected to the other end of the bending portion in an enlarged manner;

FIG. 3 is a schematic longitudinal sectional view taken along a line III-III in FIG. 2;

FIGS. 4A, 4B, 4C and 4D are schematic longitudinal sectional views showing multiple steps according to a first embodiment of a fixing structure forming method for watertightly fixing the other end portion of an exterior rubber tube, the other end portion corresponding to a base end region of an outer peripheral surface of the tip portion, to the base end region of the outer peripheral surface of the tip portion in the bending portion shown in FIG. 3;

FIGS. 5A and 5B are schematic longitudinal sectional views showing multiple steps according to a second embodiment of the fixing structure forming method for watertightly fixing the corresponding other end portion of the exterior rubber tube to the base end region of the outer surface of the tip portion in the bending portion shown in FIG. 3;

FIGS. 6A and 6B are schematic longitudinal sectional views showing multiple steps according to a third embodiment of the fixing structure forming method for watertightly fixing the corresponding other end portion of the exterior rubber tube to the base end region of the outer surface of the tip portion in the bending portion shown in FIG. 3;

FIGS. 7A and 7B are schematic longitudinal sectional views showing multiple steps according to a fourth embodiment of the fixing structure forming method for watertightly fixing the corresponding other end portion of the exterior rubber tube to the base end region of the outer surface of the tip portion in the bending portion shown in FIG. 3;

FIGS. 8A and 8B are schematic longitudinal sectional views showing multiple steps according to a fifth embodiment of the fixing structure forming method for watertightly fixing the corresponding other end portion of the exterior rubber tube to the base end region of the outer surface of the tip portion in the bending portion shown in FIG. 3;

FIG. 9A is a schematic longitudinal sectional view showing the other end portion of an exterior rubber tube which corresponds to a base end region of an outer surface of a tip portion and which is put on the base end region of the outer surface of the tip portion in a bending portion of an insertion part of a second embodiment of the endoscope according to the present invention, in a state before the other end portion is watertightly fixed to the base end region by a fixing structure;

FIG. 9B is a schematic longitudinal sectional view showing the other end portion of the exterior rubber tube in FIG. 9A in a state after the other end portion is watertightly fixed to the base end region by the fixing structure;

FIG. 10A is a schematic longitudinal sectional view showing the other end portion of an exterior rubber tube which corresponds to a base end region of an outer surface of a tip portion and which is put on the base end region of the outer surface of the tip portion in a bending portion of an insertion part of a third embodiment of the endoscope according to the present invention, in a state before the other end portion is watertightly fixed to the base end region by a fixing structure;

FIG. 10B is a schematic longitudinal sectional view showing the other end portion of the exterior rubber tube in FIG. 10A in a state after the other end portion is watertightly fixed to the base end region by the fixing structure;

FIG. 11 is a schematic longitudinal sectional view showing the other end of a bending portion and a tip portion connected to the other end in an insertion part of a fourth embodiment of the endoscope according to the present invention;

FIG. 12 is a schematic cross sectional view taken along a line XII-XII in FIG. 11;

FIG. 13 is a schematic longitudinal sectional view showing a part of the longitudinal section in FIG. 11 in an enlarged manner; and

FIG. 14 is a schematic longitudinal sectional view showing a part of the other end of a bending portion and a tip portion connected to the other end in an insertion part of a fifth embodiment of the endoscope according to the present invention in an enlarged manner.

### DETAILED DESCRIPTION OF THE INVENTION

[First Embodiment of Endoscope and First Embodiment of Forming Method of Fixing Structure for Bending Portion of Insertion Part of this Endoscope]

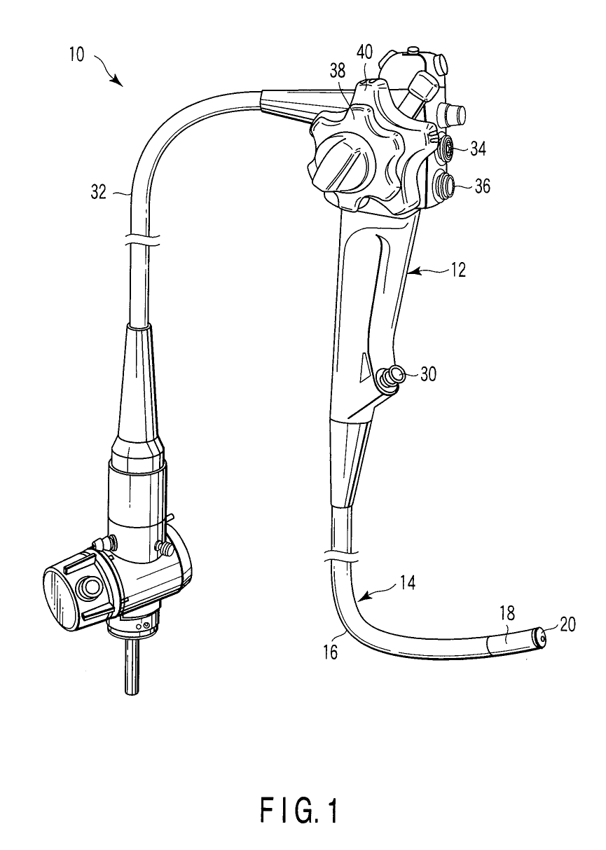

FIG. 1 schematically shows the whole structure of a first embodiment of an endoscope according to the present invention.

This endoscope is a flexible endoscope 10 for medical use, and comprises an operating part 12 and a slender insertion part 14 configured to extend from the operating part 12 and to be inserted into a body cavity of a living organism.

The insertion part 14 comprises a slender flexible tube portion 16 which has a proximal end connected to the operating part 12, a bending portion 18 which has one end connected to a distal end of the flexible tube portion 16, and a tip portion 20 which has a base end connected to the other end of the bending portion 18. The tip portion 20 is formed of metal and/or hard resin.

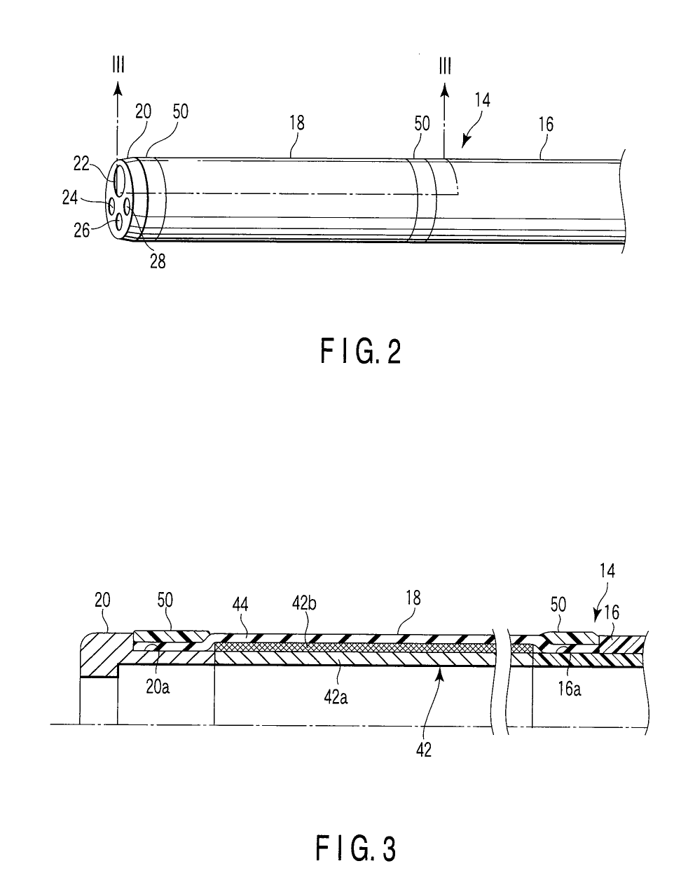

As illustrated in FIG. 2, a forceps projecting-retracting/suction opening 22, an air-feed/water-feed opening 24, an observation opening 26, and a light-projecting opening 28 are formed in a tip end surface of the tip portion 20. The observation opening 26 is watertightly covered by an object lens, and the light-projecting opening 28 is watertightly covered by a light transmission cover. In an internal space of the tip portion 22, an image pickup device is housed to correspond to the object lens of the observation opening 26.

In an internal space of each of the flexible tube portion 16 and the bending portion 18, and that of the tip portion 20 of the insertion part 14, a forceps insertion/suction pipe, an air-feed/water-feed pipe, electric wirings for the image pickup device, and a light guide tube are extended independently from the forceps projecting/suction opening 22, the air-feed/water-feed opening 24, the observation opening 26, and the light-projecting opening 28 in the tip end surface of the tip portion 20 to the proximal end of the flexible tube portion 16.

In the operating part 12, a forceps insertion opening 30 branched from the forceps insertion/suction pipe is mounted, and further a universal cord 32 is mounted. The universal cord 32 is formed by bundling a suction tube branched from the forceps insertion/suction pipe, air-feed and water-feed tubes branched from the air-feed/water-feed pipe, and the electric wirings for the image pickup device.

The suction tube, the air-feed tube, the water-feed tube, the electric wirings for the image pickup device, and the light guide tube are detachably connected to an unillustrated suction device, an unillustrated air-feed device, an unillustrated water-feed device, an unillustrated image processor for the image pickup device (including a monitor display and an image recording unit), and an unillustrated light source at an extending end of the universal cord 32.

On the operating part 12, a suction operating button 34 interposed in the suction tube and an air-feed/water-feed operating button 36 interposed in the air-feed tube and water-feed tube are mounted. Two bending portion operating knobs 38, 40 are further mounted on the operating part 12 to control a bend of the bending portion 18 of the insertion part 14 in four directions.

The bending portion 18 of the insertion part 14, as clearly shown in FIG. 3, includes a tubular bending structure 42 which can be bent to a radius smaller than the flexible tube portion 16, and an exterior rubber tube 44 which covers an outer peripheral surface of the bending structure 42. The bending structure 42 includes a bending tube 42a in which multiple rings concentrically arranged are bendably linked in four directions (cruciform directions) as a whole, and a knitted pipe 42b mounted on an outer peripheral surface of the bending tube 42a. And, the exterior rubber tube 44 covers the outer peripheral surface of the knitted pipe 42b. The knitted pipe 42b is formed by metal or chemical fibers, and both end portions of the knitted pipe 42b are fixed to both end portions of the outer peripheral surface of the bending structure 42 by a well known fixing means such as, for example, adhesives, solders, or the like.

Wires are inserted through each of the multiple rings of the bending structure 42 at four positions separated from each other at regular intervals in a circumferential direction of the ring, and one end of each wire is connected to the tip portion 20. The other ends of the four wires are connected to the two bending portion operating knobs 38, 40 of the operating part 12 via the slender flexible tube portion 16, and by operating the two bending portion operating knobs 38, 40, the bending portion 18 with the tip portion 20 can be bent in the four directions (cruciform directions) relative to the distal end of the flexible tube portion 16.

One end portion of the exterior rubber tube 44 is put on a distal end neighboring region 16a being adjacent to the distal end in the outer peripheral surface of the flexible tube portion 16 and then is watertightly fixed to the distal end neighboring region 16a by a fixing structure 50. The other end portion of the exterior rubber tube 44 is put on a base end neighboring region 20a being adjacent to the base end in the outer peripheral surface of the tip portion 20 and then is watertightly fixed to the base end neighboring region 20a by the fixing structure 50.

As is apparent from FIG. 3, an outer diameter of each of the distal end neighboring region 16a on the outer peripheral surface of the flexible tube portion 16 and the base end neighboring region 20a on the outer peripheral surface of the tip portion 20 is set to be smaller than an outer diameter of each of the other region on the outer peripheral surface of the flexible tube portion 16 and the other region on the outer peripheral surface of the tip portion 20 by a size nearly equal to the total of a thickness of the exterior rubber tube 44 and a thickness of the fixing structure 50.

In addition, an outer diameter of an intermediate portion between the one end portion and the other end portion, both being covered with the fixing structures 50, on the outer peripheral surface of the exterior rubber tube 44 of the bending portion 18 (that is, an outer diameter of the bending portion 18) is set to be substantially equal to an outer diameter of each of the other region on the outer peripheral surface of the flexible tube portion 16 and the other region on the outer peripheral surface of the tip portion 20.

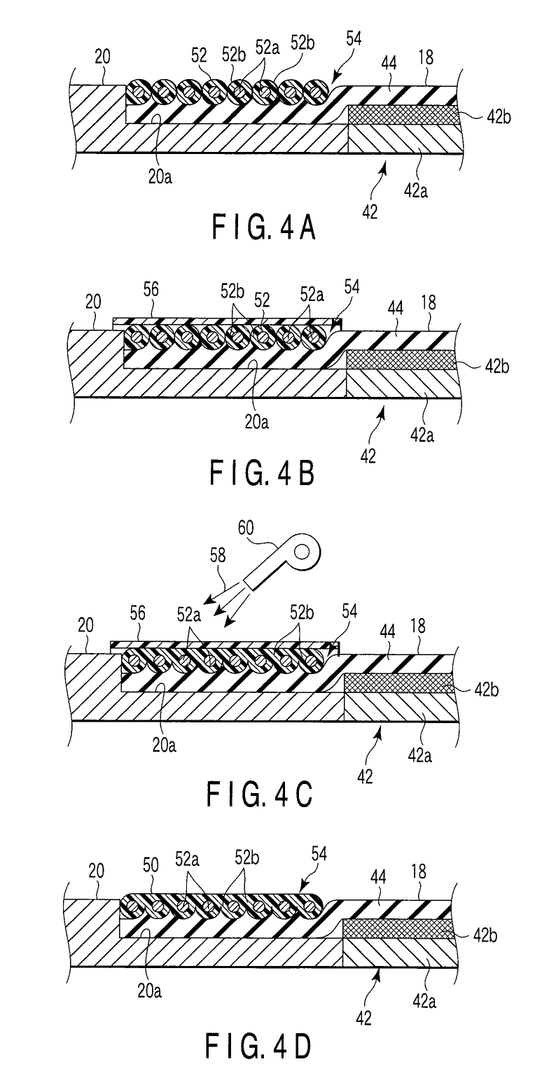

The fixing structures 50 for the one end portion and other end portion of the exterior rubber tube 44 are the same as to each other. Therefore, only a method for forming the fixing structure 50 for the other portion of the exterior rubber tube 44 will be described with reference to FIGS. 4A to 4D, and a description of a method for forming the fixing structure 50 for the one end portion of the exterior rubber tube 44 will be omitted.

FIGS. 4A to 4D schematically show multiple steps of a forming method for the fixing structure 50 according to the first embodiment of the invention.

At first, as illustrated in FIG. 4A, a string member 52 containing thermoplastic resin is wound in multiple turns with a winding force of a predetermined range on the outer circumferential surface of the other end portion of the exterior rubber tube 44 which covers the base end neighboring region 20a of the outer peripheral surface of the tip portion 22, and thereby a wound string layer 54 is formed. Multiple windings in the wound string layer 54 are adjacent to each other. In this embodiment, the thermoplastic resin of the string member 52 is of a polyolefin system, and includes for examples polypropylene and polyethylene.

In this embodiment, the string member 52 is configured by a monofilament which includes a core 52a and a cladding 52b surrounding the core 52a. A melting temperature of the cladding 52b is lower than that of the core 52a and is for example about 170° C.

Inner regions of the multiple turns in the wound string layer 54, which are directed inwards, are separated from each other to form a clearance therebetween, and the outer peripheral surface of the other end portion of the exterior rubber tube 44 enters in the clearance.

Next, as illustrated in FIG. 4B, a heat-shrinkable tube 56 is put on the whole of the outer peripheral surface of the wound string layer 54. A length of the heat-shrinkable tube 56 in a longitudinal direction of the exterior rubber tube 44 is set to be longer than a length of the wound string layer 54 in the longitudinal direction. A melting temperature of the heat-shrinkable tube 56 is higher than that of the thermoplastic resin of the string member 52. In this embodiment, the heat-shrinkable tube 56 is configured by PTFE (polytetrafluoroethylene: the shrinking temperature is about 340° C.).

Next, as illustrated in FIG. 4C, hot air 58 of a predetermined temperature is blown for a predetermined time towards the whole of an outer peripheral surface of the heat-shrinkable tube 56. Such a blowing of the hot air 58 as described above is performed by, for example, a hot air blower 60.

Such a blowing of the hot air 58 as described above fully melts outer regions of the claddings 52b of the multiple turns in the wound string layer 54, the outer regions being adjacent to the heat-shrinkable tube 56, and causes a heat shrinkage of the heat-shrinkage tube 56. The heat-shrunk tube 56 firmly fixes the fully melted outer regions of the claddings 52b of the multiple turns in the wound string layer 54 to each other, and, at the same time, smoothes the outer peripheral surface of the wound string layer 54.

The blowing of the hot air 58 of the predetermined temperature for the predetermined time does not sufficiently melt the inner regions of the claddings 52b of the multiple turns in the wound string layer 54. As a result, the clearance between the two adjacent inner regions of the multiple turns in the wound string layer 54 is maintained, and the outer peripheral surface of the other end portion of the exterior rubber tube 44 is held in these clearances. The blowing of the hot air 58 of the predetermined temperature for the predetermined time does not melt the cores 52a of the multiple turns in the wound string layer 54.

Consequently, even after the outer regions of the claddings 52b of the multiple turns in the wound string layer 54 are once fully melted and firmly fused to each other, a tightening force of the other end portion of the exterior rubber tube 44 to the base end neighboring region 20a of the outer peripheral surface of the tip portion 20 by the multiple turns in the wound string layer 54 is not decreased.

After the blowing of the hot air 58 of the predetermined temperature for the predetermined time is finished and the temperature of the wound string layer 54 falls to a room temperature, the heat-shrunk tube 56 is removed as illustrated in FIG. 4D.

The wound string layer 54, in which the outer regions of the claddings 52b of the multiple turns are firmly fused to each other and the outer peripheral surface is smoothed, provides the fixing structure 50 which achieves a watertight fixing of the other end portion of the exterior rubber tube 44 to the base end neighboring region 20a of the outer peripheral surface of the tip portion 20.

The heat shrinkable tube 56 may be configured by FEP (fluorinated ethylene propylene: the shrinking temperature is about 100° C. to about 200° C.). However, a shrinking temperature of FEP is lower than that of PTFE and different only slightly from the melting temperature of thermoplastic resin of the string member 52. Consequently, in a case that the heat-shrinkable tube 56 is configured by FEP, even after the heat-shrinkable tube 56 begins its heat-shrinkage, it is need to take further time for blowing the hot air 58 until the outer regions of the claddings 52b of the multiple turns in the wound string layer 54 are fully melted and fused to each other.

[Second Embodiment of Forming Method of Fixing Structure for Bending Portion of Insertion Part in Flexible Endoscope According to First Embodiment]

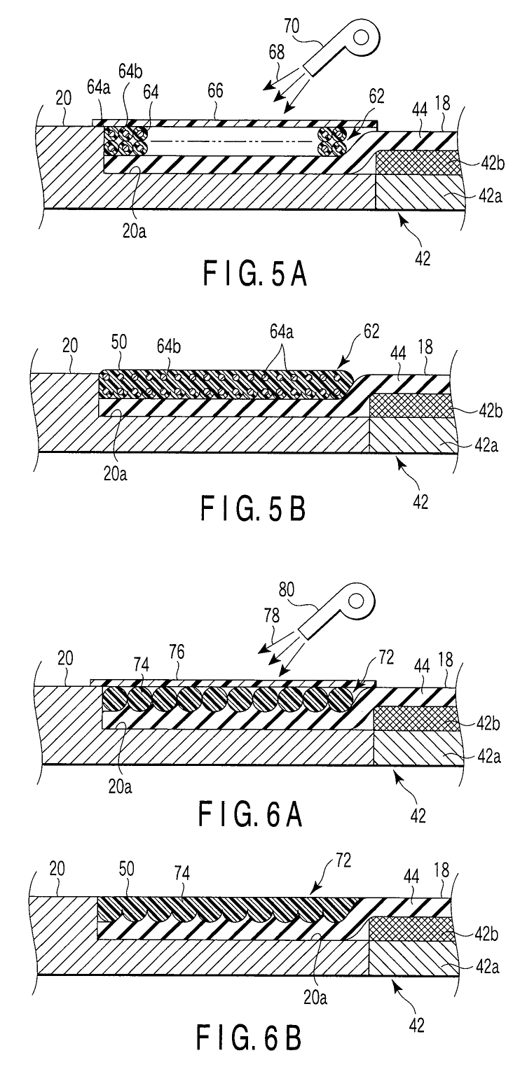

Referring now to FIGS. 5A and 5B, a second embodiment of the forming method of the fixing structure 50 for the bending portion 18 of the insertion part 14 in the flexible endoscope 10 according to the first embodiment of the present invention and described above with reference to FIGS. 1 to 3, will be described.

The second embodiment of the forming method of the fixing structure 50 is different from the first embodiment of the forming method of the fixing structure 50 described above with reference to FIGS. 4A to 4D, as follows. That is, a string member 64, which contains thermoplastic resin and which is wound as illustrated in FIG. 5A in multiple turns with a winding force of a predetermined range on the outer surface of the other end portion of the exterior rubber tube 44 put on the base end neighboring region 20a of the outer peripheral surface of the tip portion 20 and which configures a wound string layer 62, is a twisted yarn of multiple filaments. Also in this embodiment, the thermoplastic resin of the string member 64 is of the polyolefin system, and includes for example polypropylene and polyethylene.

In this embodiment, each of multiple filaments of the string member 64 includes a core 64a and a cladding 64b surrounding the core 64a. A melting temperature of the cladding 64b is lower than that of the core 64a and is for example about 170° C.

Inner regions in the multiple turns of the wound string layer 62, which direct inwards, are separated from each other to form a clearances therebetween, and the outer peripheral surface of the other end portion of the exterior rubber tube 44 is entered in these clearances.

As illustrated in FIG. 5A, a heat-shrinkable tube 66 is put on the whole of an outer peripheral surface of the wound string layer 62. A length of the heat-shrinkable tube 66 in the longitudinal direction of the exterior rubber tube 44 is set to be longer than a length of the wound string layer 62 in the longitudinal direction. A melting temperature of the heat-shrinkable tube 66 is higher than that of the thermoplastic resin of the string member 64. In this embodiment, the heat-shrinkable tube 66 is configured by PTFE (polytetrafluoroethylene: the shrinking temperature is about 340° C.).

As illustrated in FIG. 5A, hot air 68 of a predetermined temperature is blown for a predetermined time towards the whole of an outer peripheral surface of the heat-shrinkable tube 66. Such a blowing of the hot air 68 as described above is performed by, for example, a hot air blower 70.

The blowing of the hot air 68 as described above fully melts outer regions of the claddings 64b of the multiple turns in the wound string layer 62, the outer regions being adjacent to the heat-shrinkable tube 66, and causes a heat shrinkage of the heat-shrinkage tube 66. The heat-shrunk tube 66 firmly fixes the fully melted outer regions of the claddings 64b of the multiple turns in the wound string layer 62 and, at the same time, smoothes the outer peripheral surface of the wound string layer 62.

The blowing of the hot air 68 of the predetermined temperature for the predetermined time does not sufficiently melt the inner regions of the claddings 64b of the multiple turns in the wound string layer 62. As a result, the clearance between the two adjacent inner regions of the multiple turns in the wound string layer 62 is maintained, and the outer peripheral surface of the other end portion of the exterior rubber tube 44 is held in these clearances. The blowing of the hot air 68 of the predetermined temperature for the predetermined time does not melt the cores 64a of the multiple turns in the wound string layer 62.

Consequently, even after the outer regions of the claddings 64b of the multiple turns in the wound string layer 62 are fully melted and firmly fused to each other, a tightening force of the other end portion of the exterior rubber tube 44 to the base end neighboring region 20a of the outer peripheral surface of the tip portion 20 by the multiple turns in the wound string layer 62 is not decreased.

After the blowing of the hot air 68 of the predetermined temperature is finished and the temperature of the wound string layer 62 falls to a room temperature, the heat-shrunk tube 66 is removed as illustrated in FIG. 5B.

The wound string layer 62, in which the outer regions of the claddings 64b of the multiple turns are firmly fused to each other and the outer peripheral surface is smoothed, provides the fixing structure 50 which achieves the watertight fixing of the other end portion of the exterior rubber tube 44 to the base end neighboring region 20a of the outer peripheral surface of the tip portion 20.

The heat shrinkable tube 66 may be configured by FEP (fluorinated ethylene propylene: the shrinking temperature is about 100° C. to about 200° C.). However, the shrinking temperature of FEP is lower than that of PTFE and different only slightly from the melting temperature of thermoplastic resin of the string member 64. Consequently, in a case that the heat-shrinkable tube 66 is configured by FEP, even after the heat-shrinkable tube 66 begins its heat-shrinkage, it is need to take further time for blowing the hot air 68 until the outer regions of the claddings 64b of the multiple turns in the wound string layer 62 are fully melted and fused to each other.

[Third Embodiment of Forming Method of Fixing Structure for Bending Portion of Insertion Part in Endoscope According to First Embodiment]

Referring now to FIGS. 6A and 6B, a third embodiment of the forming method of the fixing structure 50 for the bending portion 18 of the insertion part 14 in the flexible endoscope 10 according to the first embodiment of the present invention and described above with reference to FIGS. 1 to 3, will be described.

The third embodiment of the forming method of the fixing structure 50 differs from the first embodiment of the forming method of the fixing structure 50 described above with reference to FIGS. 4A to 4D as follows. That is, a string member 74, which contains thermoplastic resin and which is wound as illustrated in FIG. 6A in multiple turns with a winding force of a predetermined range on the outer peripheral surface of the other end portion of the exterior rubber tube 44 put on the base end neighboring region 20a of the outer peripheral surface of the tip portion 20 and which configures a wound string layer 72, is monofilament of a single structure that does not contain any core and cladding. Also in this embodiment, the thermoplastic resin of the string member 74 is of the polyolefin system, and includes for example polypropylene and polyethylene. The melting temperature of the thermoplastic resin is about 170° C.

Inner regions of the multiple turns in the wound string layer 72, which direct inwards, are separated from each other to form a clearance therebetween, and the outer peripheral surface of the other end portion of the exterior rubber tube 44 enters in these clearances.

As illustrated in FIG. 6A, a heat-shrinkable tube 76 is put on the whole of an outer peripheral surface of the wound string layer 72. A length of the heat-shrinkable tube 76 in the longitudinal direction of the exterior rubber tube 44 is set to be longer than a length of the wound string layer 72 in the longitudinal direction. A melting temperature of the heat-shrinkable tube 76 is higher than that of the thermoplastic resin of the string member 74. In this embodiment, the heat-shrinkable tube 76 is configured by PTFE (polytetrafluoroethylene: the shrinking temperature is about 340° C.).

As illustrated in FIG. 6A, hot air 78 of a predetermined temperature is blown for a predetermined time towards the whole of an outer peripheral surface of the heat-shrinkable tube 76. Such a blowing of the hot air 78 as described above is performed by, for example, a hot air blower 80.

The blowing of the hot air 78 as described above fully melts outer regions of the multiple turns in the wound string layer 72, the outer regions being adjacent to the heat-shrinkable tube 76, and causes a heat shrinkage of the heat-shrinkage tube 76. The heat-shrunk tube 76 firmly fixes the fully melted outer regions of the multiple turns in the wound string layer 72 to each other, and, at the same time, smoothes the outer peripheral surface of the wound string layer 72.

The blowing of the hot air 78 of the predetermined temperature for the predetermined time does not fully melt inner regions of the multiple turns in the wound string layer 72. As a result, the clearance between the two adjacent inner regions of the multiple turns in the wound string layer 72 is maintained, and the outer peripheral surface of the other end portion of the exterior rubber tube 44 is held in these clearances.

Consequently, even after the outer regions of the multiple turns in the wound string layer 72 are fully melted and firmly fused to each other, a tightening force of the other end portion of the exterior rubber tube 44 to the base end neighboring region 20a of the outer peripheral surface of the tip portion 20 by the multiple turns of the wound string layer 72 is not decreased.

After the blowing of the hot air 78 of the predetermined temperature for the predetermined time is finished and the temperature of the wound string layer 72 falls to a room temperature, the heat-shrunk tube 76 is removed as illustrated in FIG. 6B.

The wound string layer 72, in which the outer regions of the multiple turns are firmly fused to each other and the outer surface is smoothed, provides the fixing structure 50 which achieves the watertight fixing of the other end portion of the exterior rubber tube 44 to the base end neighboring region 20a of the outer peripheral surface of the tip portion 20.

The heat shrinkable tube 76 may be configured by FEP (fluorinated ethylene propylene: the shrinking temperature is about 100° C. to about 200° C.). However, the shrinking temperature of FEP is lower than that of PTFE and different only slightly from the melting temperature of thermoplastic resin of the string member 74. Consequently, in a case that the heat-shrinkable tube 76 is configured by FEP, even after the heat-shrinkable tube 76 begins its heat-shrinkage, it is need to take further time for blowing the hot air 78 until the outer regions of the multiple turns in the wound string layer 72 are fully melted and fused to each other.

[Fourth Embodiment of Forming Method of Fixing Structure for Bending Portion of Insertion Part in Endoscope According to First Embodiment]

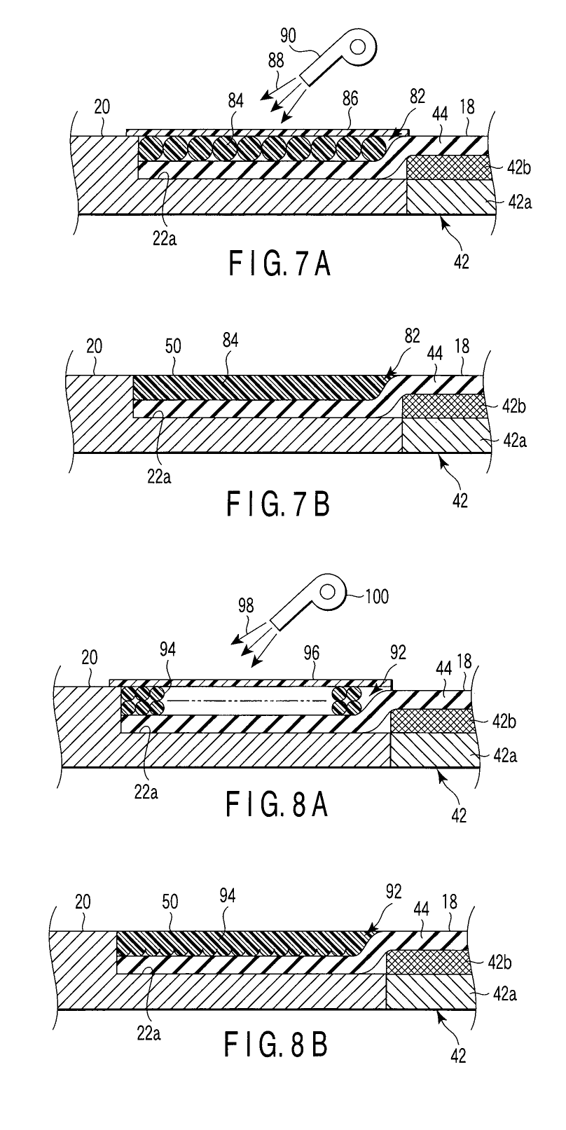

Referring now to FIGS. 7A and 7B, a fourth embodiment of the forming method of the fixing structure 50 for the bending portion 18 of the insertion part 14 in the flexible endoscope 10 according to the first embodiment of the present invention and described above with reference to FIGS. 1 to 3, will be described.

The fourth embodiment of the forming method of the fixing structure 50 differs from the first embodiment of the forming method of the fixing structure 50 described above with reference to FIGS. 4A to 4D as follows. That is, a string member 84, which contains thermoplastic resin and which is wound as illustrated in FIG. 7A in multiple turns with a winding force of a predetermined range on the outer peripheral surface of the other end portion of the exterior rubber tube 44 put on the base end neighboring region 20a of the outer peripheral surface of the tip portion 20 and which configures a wound string layer 82, is a monofilament of a single structure that does not contain any core and cladding. Also in this embodiment, the thermoplastic resin of the string member 84 is of the polyolefin system, and includes for example polypropylene and polyethylene. The melting temperature of the thermoplastic resin is about 170° C.

Inner regions of the multiple turns in the wound string layer 82, which direct inwards, are separated from each other to form a clearance therebetween, and the outer peripheral surface of the other end portion of the exterior rubber tube 44 enters in these clearances.

As illustrated in FIG. 7A, a heat-shrinkable tube 86 is put on the whole of an outer peripheral surface of the wound string layer 82. A length of the heat-shrinkable tube 86 in the longitudinal direction of the exterior rubber tube 44 is set to be longer than a length of the wound string layer 82 in the longitudinal direction. A melting temperature of the heat-shrinkable tube 86 is higher than that of the thermoplastic resin of the string member 84. In this embodiment, the heat-shrinkable tube 86 is configured by PTFE (polytetrafluoroethylene: the shrinking temperature is about 340° C.).

As illustrated in FIG. 7A, hot air 88 of a predetermined temperature is blown for a predetermined time towards the whole of an outer peripheral surface of the heat-shrinkable tube 86. Such a blowing of the hot air 88 as described above is performed by, for example, a hot air blower 90.

The blowing of the hot air 88 as described above fully melts outer regions of the multiple turns in the wound string layer 82, the outer regions directing outward and being adjacent to the heat-shrinkable tube 86, and causes a heat shrinkage of the heat-shrinkage tube 86. The heat-shrunk tube 86 firmly fixes the fully melted outer regions of the multiple turns in the wound string layer 82 to each other, and, at the same time, smoothes an outer peripheral surface of the wound string layer 82.

In this embodiment, the predetermined time of the blowing of the hot air 88 of the predetermined temperature is set to fully melt inner regions of the multiple turns in the wound string layer 82. The fully melted and firmly fixed inner regions of the multiple turns in the wound string layer 82 increase a degree of contact of the inner regions of the multiple turns with the outer peripheral surface of the other end portion of the exterior rubber tube 44 with which the inner regions of the multiple turns are in contact.

Consequently, even after the whole multiple turns including the outer regions and inner regions in the wound string layer 82 are once fully melted and firmly fused to each other, a tightening force of the other end portion of the exterior rubber tube 44 to the base end neighboring region 20a of the outer peripheral surface of the tip portion 20 by the multiple turns of the wound string layer 82 is not decreased.

After the blowing of the hot air 88 of the predetermined temperature for the predetermined time is finished and the temperature of the wound string layer 82 falls to a room temperature, the heat-shrunk tube 86 is removed as illustrated in FIG. 7B.

The wound string layer 82, in which the whole multiple turns including the outer regions and inner regions turns are firmly fused to each other and the outer peripheral surface is smoothed, provides the fixing structure 50 which achieves the watertight fixing of the other end portion of the exterior rubber tube 44 to the base end neighboring region 20a of the outer peripheral surface of the tip portion 20.

The heat shrinkable tube 86 may be configured by FEP (fluorinated ethylene propylene: the shrinking temperature is about 100° C. to about 200° C.). However, the shrinking temperature of FEP is lower than that of PTFE and different only slightly from the melting temperature of thermoplastic resin of the string member 84. Consequently, in a case that the heat-shrinkable tube 86 is configured by FEP, even after the heat-shrinkable tube 86 begins its heat-shrinkage, it is need to take further time for blowing the hot air 88 until the outer regions of the multiple turns in the wound string layer 82 are fully melted and fused to each other.

[Fifth Embodiment of Forming Method of Fixing Structure for Bending Portion of Insertion Part in Endoscope According to First Embodiment]

Referring now to FIGS. 8A and 8B, a fifth embodiment of the forming method of the fixing structure 50 for the bending portion 18 of the insertion part 14 in the flexible endoscope 10 according to the first embodiment of the present invention and described above with reference to FIGS. 1 to 3, will be described.

The fifth embodiment of the forming method of the fixing structure 50 differs from the first embodiment of the forming method of the fixing structure 50 described above with reference to FIGS. 4A to 4D as follows. That is, a string member 94, which contains thermoplastic resin and which is wound as illustrated in FIG. 8A in multiple turns with a winding force of a predetermined range on the outer peripheral surface of the other end portion of the exterior rubber tube 44 put on the base end neighboring region 20a of the outer peripheral surface of the tip portion 20 and which configures a wound string layer 92, is a twisted yarn of multiple filaments and each of the multiple filaments of the string member 94 is of a single structure that does not contain any core and cladding.

Also in this embodiment, the thermoplastic resin of the string member 94 is of the polyolefin system, and includes for example polypropylene and polyethylene. The melting temperature of the thermoplastic resin is about 170° C.

Inner regions of the multiple turns in the wound string layer 92, which direct inwards, are separated from each other to form a clearance therebetween, and the outer peripheral surface of the other end portion of the exterior rubber tube 44 enters in these clearances.

As illustrated in FIG. 8A, a heat-shrinkable tube 96 is put on the whole of an outer peripheral surface of the wound string layer 92. A length of the heat-shrinkable tube 96 in the longitudinal direction of the exterior rubber tube 44 is set to be longer than a length of the wound string layer 92 in the longitudinal direction. A melting temperature of the heat-shrinkable tube 96 is higher than that of the thermoplastic resin of the string member 94. In this embodiment, the heat-shrinkable tube 96 is configured by PTFE (polytetrafluoroethylene: the shrinking temperature is about 340° C.).

As illustrated in FIG. 8A, hot air 98 of a predetermined temperature is blown for a predetermined time towards the whole of an outer peripheral surface of the heat-shrinkable tube 96. Such a blowing of the hot air 98 as described above is performed by, for example, a hot air blower 100.

The blowing of the hot air 98 as described above fully melts outer regions of the multiple turns in the wound string layer 92, the outer regions being adjacent to the heat-shrinkable tube 96, and causes a heat shrinkage of the heat-shrinkage tube 96. The heat-shrunk tube 96 firmly fixes the fully melted outer regions of the multiple turns in the wound string layer 92 to each other, and, at the same time, smoothes an outer peripheral surface of the wound string layer 92.

The blowing of the hot air 98 of the predetermined temperature for the predetermined time does not sufficiently melt inner regions of the multiple turns in the wound string layer 92. As a result, the clearance between the two adjacent inner regions of the multiple turns in the wound string layer 92 is maintained, and the outer peripheral surface of the other end portion of the exterior rubber tube 44 is held in these clearances.

Consequently, even after the outer regions of the multiple turns in the wound string layer 92 are once fully melted and firmly fused to each other, a tightening force of the other end portion of the exterior rubber tube 44 to the base end neighboring region 20a of the outer peripheral surface of the tip portion 20 by the multiple turns in the wound string layer 92 is not decreased.

After the blowing of the hot air 98 of the predetermined temperature for the predetermined time is finished and the temperature of the wound string layer 92 falls to a room temperature, the heat-shrunk tube 96 is removed as illustrated in FIG. 8B.

The wound string layer 92, in which the outer regions of the multiple turns are firmly fused to each other and the outer surface is smoothed, provides the fixing structure 50 which achieves the watertight fixing of the other end portion of the exterior rubber tube 44 to the base end neighboring region 20a of the outer peripheral surface of the tip portion 20.

A predetermined time of the blowing of the hot air 98 of the predetermined temperature may be set to fully melt the inner regions of the multiple turns in the wound string layer 92. The fully melted and firmly fixed inner regions of the multiple turns in the wound string layer 92 increase a degree of contact of the inner regions of the multiple turns with the outer peripheral surface of the other end portion of the exterior rubber tube 44 with which the inner regions of the multiple turns are in contact.

Consequently, even after the whole multiple turns including the outer regions and inner regions in the wound string layer 92 are fully melted and firmly fused to each other, the tightening force of the other end portion of the exterior rubber tube 44 to the base end neighboring region 20a of the outer peripheral surface of the tip portion 20 by the multiple turns in the wound string layer 92 is not decreased.

The heat shrinkable tube 96 may be configured by FEP (fluorinated ethylene propylene: the shrinking temperature is about 100° C. to about 200° C.). However, the shrinking temperature of FEP is lower than that of PTFE and different only slightly from the melting temperature of thermoplastic resin of the string member 94. Consequently, in a case that the heat-shrinkable tube 96 is configured by FEP, even after the heat-shrinkable tube 96 begins its heat-shrinkage, it is need to take further time for blowing the hot air 98 until the outer regions of the multiple turns in the wound string layer 92 are fully melted and fused to each other.

[Bending Portion of Insertion Part in Endoscope According to Second Embodiment]

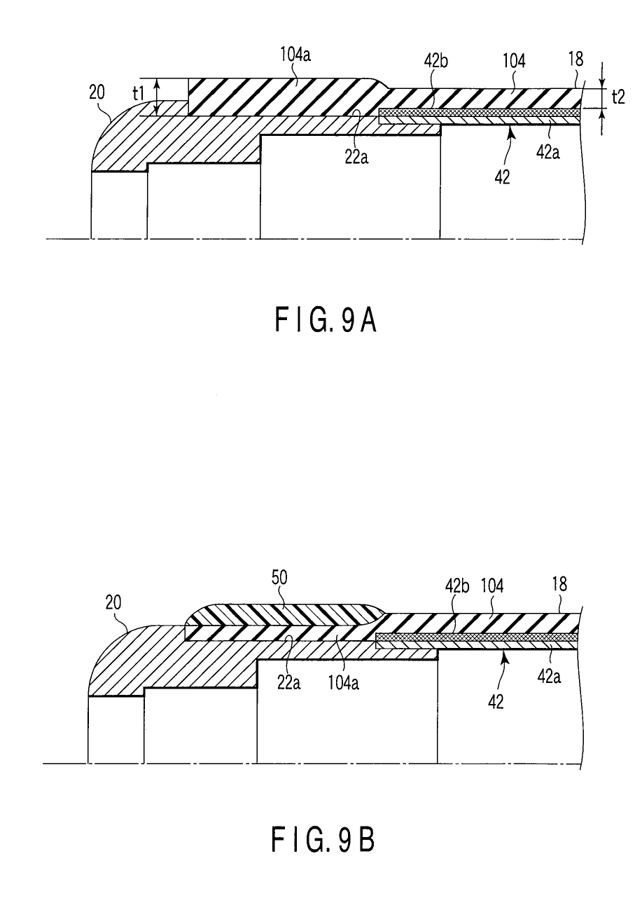

Referring now to FIGS. 9A and 9B, a bending portion 18 of an insertion part in a flexible endoscope according to a second embodiment of the present invention will be described.

The overall configuration of the flexible endoscope according to the present embodiment and excluding the both end portions of the bending portion 18 is the same as the overall configuration of the flexible endoscope 10 according to the first embodiment of the present invention and shown in FIGS. 1 to 3 and excluding the both end portions of the bending portion 18.

In the flexible endoscope according to the second embodiment, as illustrated in FIG. 9A, a thickness t1 of each of the both end portions 104a of an exterior rubber tube 104 that covers the outer peripheral surface of the bending structure 42 in the bending portion 18 of the insertion part is set to be larger than a thickness t2 of an intermediate portion between the both end portions.

As illustrated in FIG. 9A, the both end portions 104a of the exterior rubber tube 104 of the bending portion 18 are put on the distal end neighboring region 16a (refer to FIGS. 1 to 3) of the outer peripheral surface of the flexible tube portion 16 and on the base end neighboring region 20a (refer to FIGS. 1 to 3, and 9A) of the outer peripheral surface of the tip portion 20. Further, as illustrated in FIG. 9B, the both end portions 104a of the exterior rubber tube 104 of the bending portion 18 are watertightly fixed to the distal end neighboring region 16a (refer to FIGS. 1 to 3) on the outer peripheral surface of the flexible tube portion 16 and to the base end neighboring region 20a (refer to FIGS. 1 to 3, and 9A) on the outer peripheral surface of the tip portion 20 by the fixing structures 50.

The fixing structure 50 can be formed by any of the forming method of the fixing structure 50 according to the first embodiment described with reference to FIGS. 4A to 4D, the forming method of the fixing structure 50 according to the second embodiment described with reference to FIGS. 5A and 5B, the forming method of the fixing structure 50 according to the third embodiment described with reference to FIGS. 6A and 6B, the forming method of the fixing structure 50 according to the fourth embodiment described with reference to FIGS. 7A and 7B, and the forming method of the fixing structure 50 according to the fifth embodiment described with reference to FIGS. 8A and 8B.

And, setting the thickness t1 of each of the both end portions 104a of the exterior rubber tube 104 to be larger than the thickness t2 of the intermediate portion between the both end portions creates following technical advantages as compared to a case in which the thickness of each of the both end portions of the exterior rubber tube 44 used in the bending portion 18 of the insertion part 14 of the endoscope 10 of the first embodiment described with reference to FIGS. 1 to 3 is the same as that of the intermediate portion between the both end portions.

That is, when the both end portions 104a of the exterior rubber tube 104 of the bending portion 18 are put on the distal end neighboring region 16a (refer to FIGS. 1 to 3) on the outer peripheral surface of the flexible tube portion 16 and on the base end neighboring region 20a (refer to FIGS. 1 to 3, and 9A) on the outer peripheral surface of the tip portion 20, and then the fixing structure 50 is formed on each of the both end portions 104a of the exterior rubber tube 104 by any of the forming methods of the fixing structures 50 according to the first to fifth embodiments, a winding force used to wind any of the string members 52, 64, 74, 84, and 94 on each of the both end portions 104a of the exterior rubber tube 104 can be increased. By doing so, the outer diameter of each of the both thick end portions 104a of the exterior rubber tube 104 is elastically reduced, and a degree of contact of the multiple turns of any of the string members 52, 64, 74, 84, and 94 with each of the both thick end portions 104a of the exterior rubber tube 104 is increased. As a result, the watertight fixations of the both end portions 104a of the exterior rubber tube 104 of the bending portion 18 to the distal end neighboring region 16a (refer to FIGS. 1 to 3) on the outer peripheral surface of the flexible tube portion 16 and to the base end neighboring region 20a (refer to FIGS. 1 to 3, and 9A) on the outer peripheral surface of the tip portion 20 by the fixing structures 50 are still more reinforced.

By the way, according to one aspect of the present invention, it is acceptable that the thickness t1 of at least one of the both end portions 104a of the exterior rubber tube 104 is set to be larger than the thickness t2 of the intermediate portion between the both end portions.

[Bending Portion of Insertion Part in Endoscope According to Third Embodiment]

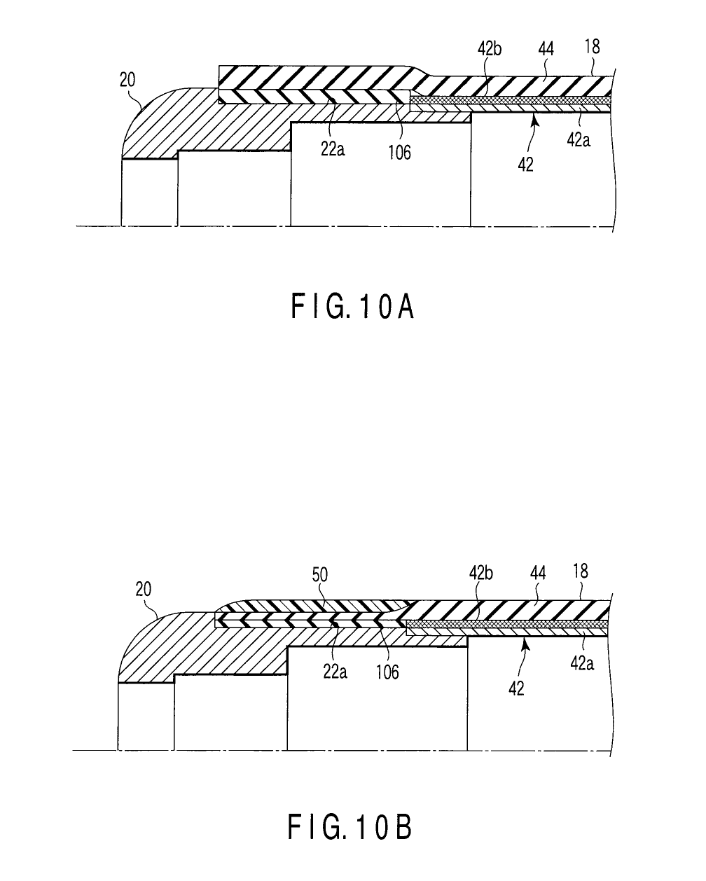

Now, referring to FIGS. 10A and 10B, a bending portion 18 of an insertion part in a flexible endoscope according to a third embodiment of the present invention will be described as follows.

The overall configuration of the flexible endoscope according to the present embodiment and excluding the both end portions of the bending portion 18 is the same as the overall configuration of the flexible endoscope 10 according to the first embodiment of the present invention and shown in FIGS. 1 to 3 and excluding the both end portions of the bending portion 18.

In the flexible endoscope according to the third embodiment, as illustrated in FIG. 10A, both end portions of an exterior rubber tube 44 that covers the outer peripheral surface of the bending structure 42 in the bending portion 18 of the insertion part are put on the distal end neighboring region 16a (refer to FIGS. 1 to 3) on the outer peripheral surface of the flexible tube portion 16 and on the base end neighboring region 20a (refer to FIGS. 1 to 3, and 10A) on the outer peripheral surface of the tip portion 20 with elastic bodies 106 made of rubber being interposed therebetween. Further, as illustrated in FIG. 10B, the both end portions of the exterior rubber tube 44 of the bending portion 18 are watertightly fixed to the distal end neighboring region 16a (refer to FIGS. 1 to 3) on the outer peripheral surface of the flexible tube portion 16 and to the base end neighboring region 20a (refer to FIGS. 1 to 3, and 10A) on the outer peripheral surface of the tip portion 20 by the fixing structures 50.

The fixing structure 50 may be formed by any of the forming method of the fixing structure 50 according to the first embodiment described with reference to FIGS. 4A to 4D, the forming method of the fixing structure 50 according to the second embodiment described with reference to FIGS. 5A and 5B, the forming method of the fixing structure 50 according to the third embodiment described with reference to FIGS. 6A and 6B, the forming method of the fixing structure 50 according to the fourth embodiment described with reference to FIGS. 7A and 7B, and the forming method of the fixing structure 50 according to the fifth embodiment described with reference to FIGS. 8A and 8B.

Interposing the elastic body 106 made of rubber between each of the both end portions of the exterior rubber tube 104 of the bending portion 18 and each of the distal end neighboring region 16a (refer to FIGS. 1 to 3) on the outer peripheral surface of the flexible tube portion 16 and the base end neighboring region 20a (refer to FIGS. 1 to 3, and 10A) on the outer peripheral surface of the tip portion 20 creates following technical advantages as compared to a case in which, as is the case of the endoscope 10 of the first embodiment described with reference to FIGS. 1 to 3, no elastic body made of rubber is interposed between each of the both end portions of the exterior rubber tube 44 used in the bending portion 18 of the insertion part 14 and each of the distal end neighboring region 16a (refer to FIGS. 1 to 3) on the outer peripheral surface of the flexible tube portion 16 and the base end neighboring region 20a (refer to FIGS. 1 to 3, and 10A) on the outer peripheral surface of the tip portion 20.

That is, when each of the both end portions of the exterior rubber tube 44 of the bending portion 18 is put on each of the distal end neighboring region 16a (refer to FIGS. 1 to 3) on the outer peripheral surface of the flexible tube portion 16 and the base end neighboring region 20a (refer to FIGS. 1 to 3, and 9A) on the outer peripheral surface of the tip portion 20 with the elastic body 106 made of rubber being interposed therebetween, and then the fixing structure 50 is formed on each of the both end portions of the exterior rubber tube 44 by any of the forming methods of the fixing structures 50 according to the first to fifth embodiments, a winding force used to wind any of the string members 52, 64, 74, 84, and 94 on each of the both end portions of the exterior rubber tube 44 can be increased. By doing so, a degree of contact of the multiple turns of any of the string members 52, 64, 74, 84, and 94 with each of the both end portions of the exterior rubber tube 44 is increased. As a result, the watertight fixations of the both end portions of the exterior rubber tube 44 of the bending portion 18 to the distal end neighboring region 16a (refer to FIGS. 1 to 3) on the outer peripheral surface of the flexible tube portion 16 and to the base end neighboring region 20a (refer to FIGS. 1 to 3, and 9A) on the outer peripheral surface of the tip portion 20 by the fixing structures 50 are still more reinforced.

By the way, according to one aspect of the present invention, it is acceptable that the elastic body 106 made of rubber is interposed between at least one of the both end portions of the exterior rubber tube 44 of the bending portion 18 and at least one of the distal end neighboring region 16a (refer to FIGS. 1 to 3) on the outer peripheral surface of the flexible tube portion 16 and the base end neighboring region 20a (refer to FIGS. 1 to 3, and 9A) on the outer peripheral surface of the tip portion 20, the former one corresponding to the latter one.

In addition, the elastic body 106 made of rubber is preferably annular. However, the elastic body 106 may not correspond to the whole area of at least one of the both end portions of the exterior rubber tube 44 in its longitudinal direction, and the elastic body 106 may partially correspond to the whole area of the at least one of the both end portions of the exterior rubber tube 44 in its longitudinal direction.

[Bending Portion and Tip Portion of Insertion Part in Endoscope According to Fourth Embodiment]

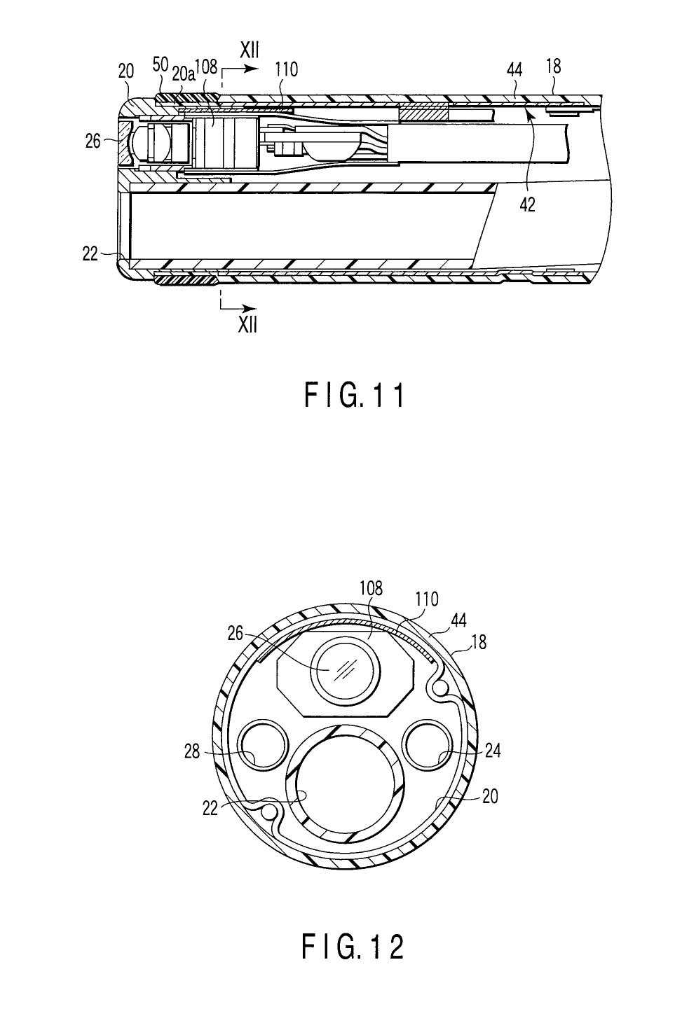

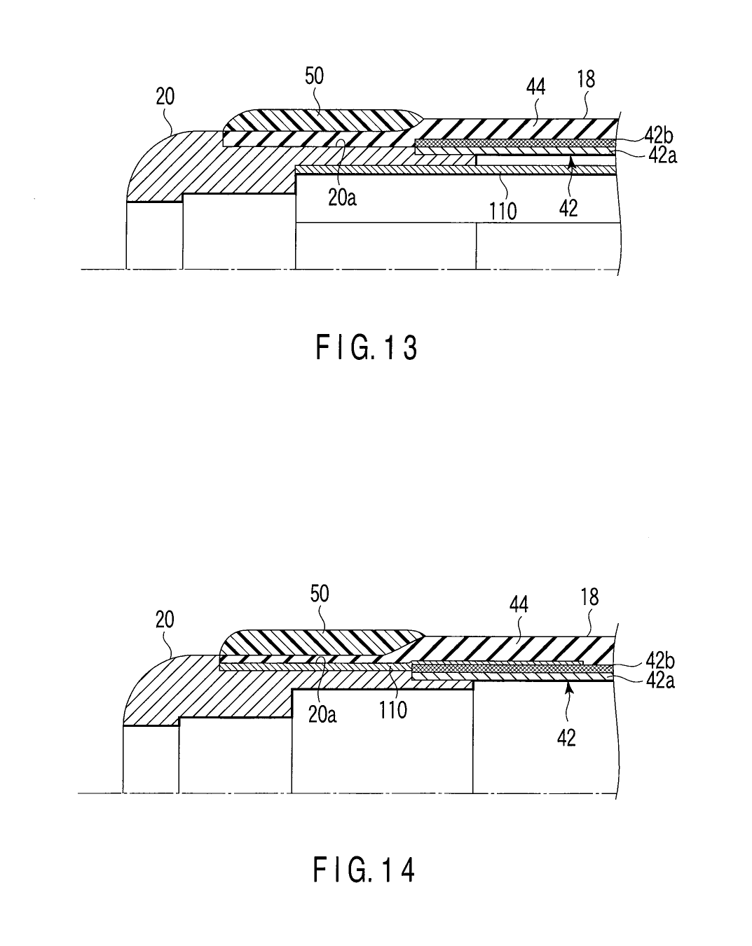

Next, referring to FIGS. 11 to 13, a bending portion 18 and a tip portion 20 of an insertion part of a flexible endoscope according to a fourth embodiment of the preset invention will be described.

The overall configuration of the flexible endoscope according to the present embodiment and excluding the base end of the tip portion 20 connected to the other end of the bending portion 18 is the same as the overall configuration of the flexible endoscope 10 according to the first embodiment of the present invention and shown in FIGS. 1 to 3 and excluding the base end of the tip portion 20 connected to the other end of the bending portion 18.

As described above about the overall configuration of the flexible endoscope 10 according to the first embodiment of the present invention with reference to FIGS. 1 to 3, in the tip portion 20 connected to the other end of the bending portion 18, an image pickup device 108 is housed to correspond to the object lens of the observation opening 26. The image pickup device 108 is located inwards relative to the base end neighboring region 20a on the outer peripheral surface of the tip portion 20 in the radial direction of the outer peripheral surface.

The base end of the tip portion 20 connected to the other end of the bending portion 18 of the insertion part in the flexible endoscope according to the fourth embodiment differs from the base end of the tip portion 20 connected to the other end of the bending portion 18 of the insertion part 14 in the flexible endoscope 10 according to the first embodiment as follows. That is, a heat insulation member 110 is installed in an inner space of the base end of the tip portion 20, in which the image pickup device 108 is housed, and the heat insulation member 110 is interposed between the image pickup device 108 and the base end neighboring region 20a on the outer peripheral surface of the base end.

The heat insulation member 110 protects the image pickup device 108 from heat generated when the fixing structure 50, which watertightly fixes the other end portion of the outer rubber tube 44 of the bending portion 18 put on the base end neighboring region 20a on the outer peripheral surface of the tip portion 20 to the base end neighboring region 20a, is formed by any of the forming method of the fixing structure 50 according to the first embodiment described with reference to FIGS. 4A to 4D, the forming method of the fixing structure 50 according to the second embodiment described with reference to FIGS. 5A and 5B, the forming method of the fixing structure 50 according to the third embodiment described with reference to FIGS. 6A and 6B, the forming method of the fixing structure 50 according to the fourth embodiment described with reference to FIGS. 7A and 7B, and the forming method of the fixing structure 50 according to the fifth embodiment described with reference to FIGS. 8A and 8B.

The heat insulation member 110 can be configured by, for example, ceramics. The heat insulation member 110 may be installed in the inner space of the base end of the tip portion 20 in such a manner that, if a component being easily affected by heat is included in various components housed in the inner space of the tip portion 20 in addition to the image pickup device 108, the heat insulation member 110 is interposed between the component being easily affected by heat and the base end neighboring region 20a on the outer peripheral surface of the tip portion 20.

[Bending Portion and Tip Portion in Insertion Part of Endoscope According to Fifth Embodiment]

Next, referring to FIG. 14, a bending portion 18 and a tip portion 20 in an insertion part of a flexible endoscope according to a fifth embodiment of the preset invention will be described.

The overall configuration of the flexible endoscope according to the present embodiment and excluding a base end of the tip portion 20 connected to the other end of the bending portion 18 is the same as the overall configuration of the flexible endoscope 10 according to the first embodiment of the present invention and shown in FIGS. 1 to 3 and excluding the base end of the tip portion 20 connected to the other end of the bending portion 18.

The base end of the tip portion 20 connected to the other end of the bending portion 18 of the insertion part in the flexible endoscope according to the fifth embodiment differs from the base end of the tip portion 20 connected to the other end of the bending portion 18 of the insertion part 14 in the flexible endoscope 10 according to the first embodiment as follows. That is, a heat-insulation member 110 is installed on the base end neighboring region 20a on the outer peripheral surface of the tip portion 20.

In this embodiment, the other end portion of the exterior rubber tube 44 of the bending portion 18 is put on the base end neighboring region 20a on the outer peripheral surface of the tip portion 20 with the heat insulation member 110 being interposed therebetween. The heat insulation member 110 protects various components housed in the inner space of the base end of the tip portion 20, in particular, components being comparatively and easily affected by heat such as the image pickup device 108 (refer to FIGS. 11 and 12) included in the various components, from heat generated when the fixing structure 50, which watertightly fixes the other end portion of the exterior rubber tube 44 to the base end neighboring region 20a, is formed by any of the forming method of the fixing structure 50 according to the first embodiment described with reference to FIGS. 4A to 4D, the forming method of the fixing structure 50 according to the second embodiment described with reference to FIGS. 5A and 5B, the forming method of the fixing structure 50 according to the third embodiment described with reference to FIGS. 6A and 6B, the forming method of the fixing structure 50 according to the fourth embodiment described with reference to FIGS. 7A and 7B, and the forming method of the fixing structure 50 according to the fifth embodiment described with reference to FIGS. 8A and 8B.

The heat insulation member 110 can be configured by, for example, ceramics. The heat insulation member 110 need not be installed on the whole area of the base end neighboring region 20a on the outer surface of the tip portion 20, as long as it can ensure that the other end portion of the exterior rubber tube 44 put on the base end neighboring region 20a on the outer peripheral surface of the tip portion 20 with the heat insulation member 110 being interposed therebetween can be watertightly fixed to the base end neighboring region 20a on the outer peripheral surface of the tip portion 20 by the fixing structure 50. The heat insulation member 110 may be installed only to a portion of the base end neighboring region 20a on the outer peripheral surface of the tip portion 20, the portion corresponding to elements being comparatively and easily affected by heat such as the image pickup device 108 (refer to FIGS. 11 and 12) included in various components housed in the inner space of the base end of the tip portion 20.

In the various embodiments of the fixing structure 50 described above, in order to melt the outer region or both of the outer region and the inner region in each of the wound string layers 54, 62, 72, 82, and 92, each of the hot air 58, 68, 78, 88, and 98 blew from the hot air blowers 60, 70, 80, 90, and 100 are blew to each of the heat-shrinkable tubes 56, 66, 76, 86, and 96, put on each of the outer peripheral surfaces of the wound string layers 54, 62, 72, 82, and 92. However, in a case that the above-mentioned melting and shaping of the outer surfaces of the melted outer regions of the wound string layers 54, 62, 72, 82, and 92 are properly executed, the heat-shrinkable tubes 56, 66, 76, 86, and 96 may be omitted.

Furthermore, in a case that above-mentioned melting and shaping of the outer surfaces of the melted outer regions of the wound string layers 54, 62, 72, 82, and 92 can be properly executed without affecting on components being comparatively and easily affected by heat such as the image pickup device 108 (refer to FIGS. 11 and 12) and included in various components housed in the inner space of the base end of the tip portion 20, various well known heating means other than hot air, for example, entering the wound string layers 54, 62, 72, 82, and 92 into a heating furnace, may be adopted.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An insertion part configured to extend from an operating part in an endoscope and to be inserted into a body cavity of a living organism, comprising:
a slender flexible tube portion having a proximal end and a distal end, the proximal end being connected to the operating part;
a bending portion having one end and the other end, the one end being connected to the distal end of the flexible tube portion; and
a tip portion having a base end and a tip end, the base end being connected to the other end of the bending portion,
wherein the bending portion includes: a tubular bending structure bendable to a radius smaller than the flexible tube portion; an exterior rubber tube which has one end portion put on a distal end neighboring region being adjacent to the distal end on an outer peripheral surface of the flexible tube portion and the other end portion put on a base end neighboring region being adjacent to the base end on an outer peripheral surface of the tip portion, and which covers an outer peripheral surface of the bending structure; and fixing structures which watertightly fix the one end portion and the other end portion of the exterior rubber tube to the distal end neighboring region of the outer peripheral surface of the flexible tube portion and the base end neighboring region of the outer peripheral surface of the tip portion, and
the fixing structure for at least one of the one end portion and the other end portion of the exterior rubber tube includes a string member containing thermoplastic resin and wound in multiple turns to be adjacent to each other on the outer peripheral surface of the at least one of the one end portion and the other end portion of the exterior rubber tube, and at least outer regions directed to an outside in the multiple turns of the string member on the outer peripheral surface of the at least one of the one end portion and the other end portion of the exterior rubber tube are melted and firmly fused to each other.

2. The insertion part according to claim 1, wherein the string member of the fixing structure is monofilament.

3. The insertion part according to claim 1, wherein the string member of the fixing structure is a twisted yarn of a plurality of filaments.

4. The insertion part according to claim 1, wherein the string member of the fixing structure includes a core and a cladding surrounding the core, and a melting point of the cladding is lower than that of the core.

5. The insertion part according to claim 1, wherein the thermoplastic resin of the string member of the fixing structure is of a polyolefin system.

6. The insertion part according to claim 1, wherein inner regions directed inwards in the multiple turns of the string member are separated from each other to form a clearance therebetween, and the outer peripheral surface of the at least one of the one end portion and the other end portion of the exterior rubber tube is entered into the clearance.

7. The insertion part according to claim 1, wherein inner regions directed inwards in the multiple turns of the string member on the outer peripheral surface of the at least one of the one end portion and the other end portion of the exterior rubber tube are melted and firmly fused to each other.

8. The insertion part according to claim 1, wherein an outer diameter of the at least one of the one end portion and the other end portion of the exterior rubber tube is set to be larger than that of an intermediate portion of the exterior rubber tube between the one end portion and the other end portion, and
the outer diameter of the at least one of the one end portion and the other end portion of the exterior rubber tube is elastically reduced by winding the string member in multiple turns on the outer peripheral surface of the at least one of the one end portion and the other end portion of the exterior rubber tube and by melting the outer regions of the multiple turns of the string member on the outer peripheral surface of the at least one of the one end portion and the other end portion of the exterior rubber tube to be firmly fused to each other.

9. The insertion part according to claim 1, wherein a rubber elastic member is interposed in at least one part between an inner peripheral surface of the at least one of the one end portion and the other end portion of the exterior rubber tube, and at least one of the distal end neighboring region of the outer surface of the flexible tube portion and the base end neighboring region of the outer surface of the tip portion corresponding to the inner peripheral surface, and
a thickness of the rubber elastic member is elastically reduced by winding the string member in multiple turns on the outer peripheral surface of the at least one of the one end portion and the other end portion of the exterior rubber tube and by melting the outer regions of the multiple turns of the string member on the outer peripheral surface of the at least one of the one end portion and the other end portion of the exterior rubber tube to be firmly fused to each other.

10. The insertion part according to claim 1, wherein a heat insulation member is interposed in at least one part which is inward from the at least one inner peripheral surface.

11. An endoscope comprising:
an operating part; and
an insertion part configured to extend from the operating part and to be inserted into a body cavity of a living organism,
the insertion part comprising:
a slender flexible tube portion having a proximal end and a distal end, the proximal end being connected to the operating part;
a bending portion having one end and the other end, the one end being connected to the distal end of the flexible tube portion; and
a tip portion having a base end and a tip end, the base end being connected to the other end of the bending portion,
wherein the bending portion includes: a tubular bending structure bendable to a radius smaller than the flexible tube portion; an exterior rubber tube which has one end portion put on a distal end neighboring region being adjacent to the distal end on an outer peripheral surface of the flexible tube portion and the other end portion put on a base end neighboring region being adjacent to the base end on an outer peripheral surface of the tip portion, and which covers an outer peripheral surface of the bending structure; and fixing structures which watertightly fix the one end portion and the other end portion of the exterior rubber tube to the distal end neighboring region of the outer peripheral surface of the flexible tube portion and the base end neighboring region of the outer peripheral surface of the tip portion, and
the fixing structure for at least one of the one end portion and the other end portion of the exterior rubber tube includes a string member containing thermoplastic resin and wound in multiple turns to be adjacent to each other on the outer peripheral surface of the at least one of the one end portion and the other end portion of the exterior rubber tube, and at least outer regions directed to an outside in the multiple turns of the string member on the outer peripheral surface of the at least one of the one end portion and the other end portion of the exterior rubber tube are melted and firmly fused to each other.

12. The endoscope according to claim 11, wherein inner regions directed inwards in the multiple turns of the string member are separated from each other to form a clearance therebetween, and the outer peripheral surface of the at least one of the one end portion and the other end portion of the exterior rubber tube is entered into the clearance.

13. The endoscope according to claim 11, wherein an outer diameter of the at least one of the one end portion and the other end portion of the exterior rubber tube is set to be larger than that of an intermediate portion of the exterior rubber tube between the one end portion and the other end portion, and
the outer diameter of the at least one of the one end portion and the other end portion of the exterior rubber tube is elastically reduced by winding the string member in multiple turns on the outer peripheral surface of the at least one of the one end portion and the other end portion of the exterior rubber tube and by melting the outer regions of the multiple turns of the string member on the outer peripheral surface of the at least one of the one end portion and the other end portion of the exterior rubber tube to be firmly fused to each other.

14. The endoscope according to claim 11, wherein a rubber elastic member is interposed in at least one part between an inner peripheral surface of the at least one of the one end portion and the other end portion of the exterior rubber tube, and at least one of the distal end neighboring region of the outer surface of the flexible tube portion and the base end neighboring region of the outer surface of the tip portion corresponding to the inner peripheral surface, and
a thickness of the rubber elastic member is elastically reduced by winding the string member in multiple turns on the outer peripheral surface of the at least one of the one end portion and the other end portion of the exterior rubber tube and by melting the outer regions of the multiple turns of the string member on the outer peripheral surface of the at least one of the one end portion and the other end portion of the exterior rubber tube to be firmly fused to each other.

15. The endoscope according to claim 11, wherein a heat insulation member is interposed in at least one part which is inward from the at least one inner peripheral surface.

16. A forming method of a fixing structure for a bending portion of an insertion part of an endoscope,
the endoscope comprising an operating part, and an insertion part configured to extend from the operating part and to be inserted into a body cavity of a living organism,
the insertion part comprising: a slender flexible tube portion having a proximal end and a distal end, the proximal end being connected to the operating part; a bending portion having one end and the other end, the one end being connected to the distal end of the flexible tube portion; and a tip portion having a base end and a tip end, the base end being connected to the other end of the bending portion,
the bending portion including: a tubular bending structure bendable to a radius smaller than the flexible tube portion; an exterior rubber tube which has one end portion put on a distal end neighboring region being adjacent to the distal end on an outer peripheral surface of the flexible tube portion and the other end portion put on a base end neighboring region being adjacent to the base end on an outer peripheral surface of the tip portion, and which covers an outer peripheral surface of the bending structure; and fixing structures which watertightly fix the one end portion and the other end portion of the exterior rubber tube to the distal end neighboring region of the outer peripheral surface of the flexible tube portion and the base end neighboring region of the outer peripheral surface of the tip portion, and
the forming method comprising:
winding a string member containing thermoplastic resin in multiple turns to be adjacent to each other on the outer peripheral surface of the at least one of the one end portion and the other end portion of the exterior rubber tube, and
melting at least outer regions directed to an outside in the multiple turns of the string member on the outer peripheral surface of the at least one of the one end portion and the other end portion of the exterior rubber tube, and firmly fusing to each other.

17. The forming method of a fixing structure according to claim 16, further comprising
separating inner regions directed inwards in the multiple turns of the string member on the outer peripheral surface of the at least one of the one end portion and the other end portion of the exterior rubber tube from each other to form a clearance therebetween, and
entering the outer peripheral surface of the at least one of the one end portion and the other end portion of the exterior rubber tube into the clearance.

18. The forming method of a fixing structure according to claim 16, further comprising
melting inner regions directed inwards in the multiple turns of the string member on the outer peripheral surface of the at least one of the one end portion and the other end portion of the exterior rubber tube, and firmly fusing to each other.

19. The forming method of a fixing structure according to claim 16, wherein melting the at least outer regions in the multiple turns of the string member and mutually fusing to each other includes blowing hot air to the at least the outer regions of the multiple turns of the string member.

20. The forming method of a fixing structure according to claim 16, further comprising putting a heat-shrinkable tube on the string member containing thermoplastic resin and wound in multiple times to be adjacent to each other on the outer peripheral surface of at least one of the one end portion and the other end portion of the exterior rubber tube,
wherein a melting temperature of the heat-shrinkable tube is higher than that of the thermoplastic resin of the string member.
